(19) United States
(12) Patent Application Publication
(10) Pub. No.: US 2023/0053056 A1
(43) Pub. Date: Feb. 16, 2023

(54) PERSONAL CARE COMPOSITION

(71) Applicant: The Procter & Gamble Company, Cincinnati, OH (US)

(72) Inventors: Sean Michael Renock, Loveland, OH (US); Jennifer Mary Marsh, Deerfield Township, OH (US); Howard David Hutton, III, Oregonia, OH (US)

(21) Appl. No.: 17/874,589

(22) Filed: Jul. 27, 2022

Related U.S. Application Data
(60) Provisional application No. 63/227,106, filed on Jul. 29, 2021.

(51) Int. Cl. A61K 8/44; A61Q 5/00; A61K 8/37; A61K 8/92; A61Q 5/02; A61Q 5/12

(52) U.S. Cl. CPC A61K 8/44; A61Q 5/006; A61K 8/375; A61K 8/922; A61Q 5/02; A61Q 5/12; A61K 2800/30; A61K 2800/34

(57) ABSTRACT

A minimal ingredient personal care composition containing 9 wt % to 16 wt % amino acid surfactant, 3 wt % to 10 wt % glyceryl ester, 0.2 wt % to 6 wt % triglyceride oil, a liquid carrier. The personal care composition is substantially free of sulfated surfactants, has a pH of 4.5 to 6 and a yield stress of 0.5 to 10 Pa. The personal care composition can be a shampoo that delivers suitable cleaning, color retention and/or conditioning benefits, while also being sulfate free.

### FIELD

The present disclosure generally relates to personal care compositions free of sulfated surfactants that deliver natural oils to the hair for dry conditioning

### BACKGROUND

Natural conditioning oils have historically been added to shampoo formulation at very low levels due to difficulty in stabilization at higher concentration within the micellar containing shampoos. Consumers are looking for natural conditioning alternatives to silicone to deliver dry conditioning benefits to the hair and skin after shampooing. Using higher levels of natural oils and maintaining stability is typically needed to achieve similar or better conditioning benefits to silicone. The invention identifies a minimal ingredient system that helps deliver the oils effectively and provides stability vs. micellar non structured shampoo systems by leveraging the unique combination of amino acid surfactant and glyceryl ester to generate a structured surfactant phase that can suspend the natural conditioning oils. This structured surfactant phase creates a low shear yield stress by which the natural oils at high levels can be suspended, stabilized, and delivered to the hair and scalp for conditioning benefits.

### SUMMARY

A personal care composition comprising from about 9 wt % to about 16 wt % amino acid surfactant; from about 3 wt % to about 10 wt % glyceryl ester; from about 0.2 wt % to about 6 wt % triglyceride oil; a liquid carrier; and wherein the personal care composition is substantially free of sulfated surfactants, has a pH of from about 4.5 to about 6 and a low shear yield stress of from about 0.5 to about 10 Pa.

### BRIEF DESCRIPTION OF THE DRAWINGS

FIG. 1 shows a DIC light microscopy image of a personal care composition with no glyceryl caprylate caprate.

FIG. 2 shows a DIC light microscopy image of a personal care composition made according to Example 2.

FIG. 3 shows a DIC light microscopy image of a personal care composition made according to Example 6.

### DETAILED DESCRIPTION

In all embodiments of the present disclosure, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at about 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “charge density,” as used herein, refers to the ratio of the number of positive charges on a polymer to the molecular weight of said polymer.

The term “comprising,” as used herein, means that other steps and other ingredients which do not affect the end result can be added. This term encompasses the terms “consisting of” and “consisting essentially of.” The compositions and methods/processes of the present disclosure can comprise, consist of, and consist essentially of the elements and limitations of the invention described herein, as well as any of the additional or optional ingredients, components, steps, or limitations described herein.

The term “personal care composition” as used herein, may be presented in typical personal care formulations. They may be in the form of solutions, dispersion, emulsions, powders, talcs, encapsulated, spheres, spongers, solid dosage forms, foams, and other delivery mechanisms. The compositions described herein may be hair tonics, leave-on hair products such as treatment, and styling products, rinse-off hair products such as shampoos, pre-wash product, co-wash product, and personal cleansing products, and treatment products; and any other form that may be applied to hair or skin.

The term “polymer,” as used herein, includes materials whether made by polymerization of one type of monomer or made by two (i.e., copolymers) or more types of monomers.

The term “suitable for application to human hair,” as used herein, means that the personal care compositions or components thereof, are acceptable for use in contact with human hair and the scalp and skin without undue toxicity, incompatibility, instability, allergic response, and the like.

The term “water soluble,” as used herein, means that the material is soluble in water. In certain embodiments, the material can be soluble at 25° C. at a concentration of 0.1% by weight of the water solvent, in certain embodiments at 1% by weight of the water solvent, in certain embodiments at 5% by weight of the water solvent, and in certain embodiments at 15% or more by weight of the water solvent.

The terms “sulfate free” and “substantially free of sulfates” means essentially free of sulfate-containing compounds except as otherwise incidentally incorporated as minor components. Sulfate free contains no detectable sulfated surfactants.

The term “sulfated surfactants” means surfactants which contain a sulfate group. The term “substantially free of sulfated surfactants” means essentially free of surfactants containing a sulfate group except as otherwise incidentally incorporated as minor components.

### Personal Care Compositions

As will be described herein, a personal care composition is disclosed which exhibits excellent cleaning and conditioning qualities without the use, or inclusion, of a sulfated surfactant or a silicone oil.

Rinse off personal care compositions such as sulfate free shampoos have historically been difficult to thicken, stabilize, and deposit levels of triglyceride oils sufficient to deliver dry conditioning, without polymeric thickeners or suspending agents such as carbomer, EGDS or thixotropic thickeners (e.g., trihydroxystearate). It has been found that personal care compositions that deliver effective levels of triglyceride oils and are sulfate free can be thickened, and stabilized, equal to or greater than sulfate containing personal care compositions. This can be accomplished via the formation of a structured surfactant phase, formed when a sulfate-free amino based surfactant is combined with a glyceryl ester. It has been also been found that triglyceride oils can be suspended by the structured surfactant phase and deposited efficiently via coacervate formation to the hair, scalp, and/or skin. Coacervate aided delivery in this system is efficient and can deliver the insoluble triglyceride oils. The coacervate formation can be further aided by the presence of a cationic polymer.

Examples of a structured surfactant phase include, but are not limited to, unilamellar and multilamellar vesicles. A unilamellar vesicle is a spherical chamber, bounded by a single bilayer of an amphiphilic lipid or a mixture of such lipids, containing an aqueous solution inside the chamber. A multilamellar vesicle contains multiple spherical chambers, bound by multiple bilayers of an amphiphilic lipid or a mixture of such lipids, containing an aqueous solution inside the chamber. Oligovesicular vesicles are spherical chambers bound by a single bilayer of amphiphilic lip or a mix of such lipids which contains an aqueous solution inside the chamber in addition to other independent spherical vesicles within the main vesicle.

Vesicles typically form upon pH adjustment. The formula will transition from semi transparent to white. Vesicles can have the structure of unilamellar, multilamellar and oligovesicles.

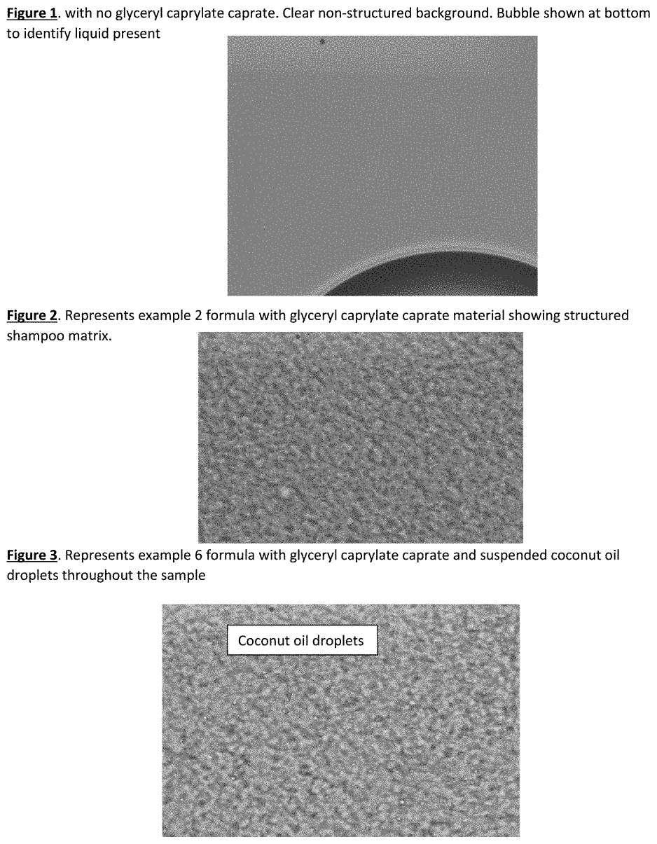

FIG. 1 shows a DIC light microscopy image of a personal care composition having surfactant and no glyceryl caprylate caprate. It shows a clear non-structured surfactant phase background. Bubbles shown at the bottom to identify the liquid present. FIG. 2 shows a DIC light microscopy image of a personal care composition made according to Example 2 having the surfactant and glyceryl caprylate caprate and forming a structured surfactant phase. FIG. 3 shows a DIC light microscopy image of a personal care composition made according to example 6 formula having surfactant and glyceryl caprylate caprate and forming the structured surfactant phase. Also FIG. 3 shows suspended coconut oil droplets.

The personal care composition can further comprise a cationic deposition polymer.

The personal care compositions described herein can be free of sulfated surfactants. Additionally the amino acid surfactant and the glyceryl caprylate caprate can be included at a ratio of 2:1. The personal care compositions described herein can be substantially free of silicones. Further, the personal care composition may comprise less than 6 wt % added salts, alternatively less than about 3 wt % of added salts, alternatively from about 0 wt % to about 6 wt % added salts.

The personal care composition has a low shear yield stress value of from about 0.5 Pa to about 10 Pa, alternatively from about 1 to about 10 Pa. This low shear yield stress value enables the stable inclusion of triglyceride oils. Further, the personal care composition can be stable at 5° C. or 40° C., alternatively at both 5° C. or 40° C.

The personal care composition has a pH of from about 4.5 to about 6, alternatively from about 4.5 to about 5.5.

It may be consumer desirable to have a personal care composition, such as a shampoo, with a minimal level of ingredients. The personal care composition described herein allows for a minimized amount of ingredients without polymeric thickeners or suspending agents such as carbomer, EGDS or thixcin etc. A personal care composition in the form of a shampoo as described herein may consist of the following 9 ingredients: water, sodium cocoyl alaninate, glyceryl caprylate/caprate, cationic polymer, triglyceride oil, sodium benzoate, citric acid, perfume, and sodium chloride. It is understood that perfumes can be formed from one or more fragrances. Alternatively, the personal care composition can comprise 8 ingredients, in particular if it is fragrance free.

### Detersive Surfactant

The personal care compositions described herein can include one or more detersive surfactants. As can be appreciated, surfactants provide a cleaning benefit to soiled hair and hair follicles by facilitating the removal of oil and other soil components from the soiled hair. Surfactants generally facilitate such cleaning due to their amphiphilic nature which allows for the surfactants to break up, and form micelles around, oil and other soils in the hair which can then be rinsed out, thereby removing them from the hair.

The detersive surfactant used in the personal care compositions described herein is an amino acid surfactant selected from the group sodium cocoyl alaninate, sodium cocoyl glycinate, sodium cocoyl sarcosinate, disodium cocoyl glutamate, and sodium lauroyl glutamate. The amino acid surfactant can also be selected from the group potassium cocoyl glycinate, potassium lauroyl glycinate, potassium lauroyl sarcosinate, potassium lauroyl glutamate, potassium lauroyl alaninate, potassium cocoyl alaninate, sodium cocoyl methyl beta-alanine, Sodium Myristoyl Methyl Beta-Alanine, Potassium Lauroyl Methyl Beta-Alanine, Sodium Cocoyl (Arginine/AsparticAcid/GlutamicAcid/Isoleucine/Leucine/Lysine/Phenylalanine/Proline/Serine/Threonine/Valine), Sodium Cocoyl/Stearoyl (Alanine/Arginine/Asparagine/Aspartic Acid/GlutamicAcid/Glutamine/Glycine/Histidine/Isoleucine/Leucine/Lysine/Phenylalanine/Proline/Serine/Threonine/Tyrosine/Valine), SodiumBehenoyl/Cocoyl (Alanine/Arginine/Asparagine/AsparticAcid/GlutamicAcid/Glycine/Isoleucine/Leucine/Lysine/Methionine/Phenylalanine/Proline/Serine/Threonine/Tryptophan/Tyrosine/Valine,PotassiumOlivoyl (Alanine/Arginine/Asparagine/AsparticAcid/GlutamicAcid/Glycine/Isoleucine/Leucine/Lysine/Methionine/Phenylalanine/Proline/Serine/Threonine/Tryptophan/Tyrosine/Valine), PotassiumLauroyl/Olivoyl (Alanine/Arginine/Asparagine/AsparticAcid/GlutamicAcid/Lycine/Isoleucine/Leucine/Lysine/Methionine/Phenylalanine/Proline/Serine/Threonine/Tryptophan/Tyrosine/Valine).

The personal care composition comprises from about 5 wt % to about 30 wt % of amino acid surfactant, from about 7 wt % to about 15, wt % of amino acid surfactant, alternatively from about 10 wt % to about 20 wt % of amino acid surfactant, alternatively from about 9 wt % to about 12 wt % of amino acid surfactant, and alternatively from about 10 wt % to about 12 wt % of amino acid surfactant. The amino acid surfactant can be sodium cocoyl alaninate, this surfactant is an anionic amino acid (Alanine) and coconut fatty acid derived surfactant from nature. This surfactant is sulfate free. The material is biodegradable, hypoallergenic, mild to skin and eye. Sodium cocoyl alaninate delivers mild cleansing which imparts pleasant moisturizing feel after drying. Sodium cocoyl alaninate can be sourced from Ajinomoto and Sino Lion.

### Cosurfactant

The composition may comprise from about 0.01 wt % to about 5 wt % of a co-surfactant, wherein the co-surfactant is a zwitterionic surfactant. The zwitterionic surfactant is a derivative of aliphatic quaternary ammonium, phosphonium, and sulfonium compounds, in which the aliphatic radicals can be straight or branched chain, and wherein 25 one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, cocosultaine, lauratnidopropyl betaine, lauryl betaine, lauryl hydroxysultaine, lauryl5sultaine, and mixtures thereof.

A suitable zwitterionic surfactant is lauryl hydroxysultaine. The zwitterionic surfactant can be selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

The co-surfactant can be a zwitterionic surfactant, wherein the zwitterionic surfactant is selected from the group consisting of: lauryl hydroxysultaine, cocamidopropyl hydroxysultaine, coco-betaine, coco-hydroxysuitaine, coco-sultaine, lauryl betaine, lauryl sultaine, and mixtures thereof.

### Triglyceride Oils

The personal care composition comprises from about 0.2 wt % to about 6 wt %, alternatively from about 0.5 wt % to about 6 wt %, alternatively from about 0.5 wt % to about 5 wt %, alternatively from about 0.5 wt % to about 3 wt % of a triglyceride oils which is an oil derived from nuts, beans, seeds etc. which contain greater than about 80 wt % triglyceride. A triglyceride is an ester derived from glycerol and three fatty acids. The triglyceride oil may comprise one or more saturated or (poly)unsaturated hydrocarbon chains having from 8 to 24 carbon atoms. All natural oils contain a mixture of triglycerides with different chain lengths and degrees of unsaturation of the fatty acids. The triglyceride oil may comprise coconut oil, sunflower seed oil, castor oil, almond oil, avocado oil, crambe oil, grapeseed oil, wheat germ oil, rice bran oil, macadamia seed oil, palm oil, evening primrose oil, soy bean oil, corn oil, tea leaf oil, hemp seed oil, olive oil, moringa oil, argan oil, apricot oil, baobab oil, batana oil, cumin oil, sesame oil, canola oil, coffee seed oil, dongbaek oil, marula oil, meadowfoam oil, oat kernel oil, seabuckthorn oil, tamanu oil, date oil and combinations thereof.

Additionally, the triglyceride oil may comprise a butter including shea, mango, cocoa and combinations thereof.

### Glyceryl Ester

The personal care composition comprises from about 5 wt % to about 8 wt %, from about from about 3 wt % to about 10 wt %, from about 4 wt % to about 10 wt %, from about 5 wt % to about 6 wt % Glyceryl Ester. Suitable glyceryl esters can be selected from glyceryl caprylate, glyceryl caprate, Glyceryl Caprylate/Caprate, and mixtures thereof. Other glyceryl esters can be selected containing C8-C10 mono-di- and tri-glycerides which are different from C8-C10 mono-di caprylate 1,2,3-propanetriol. Glyceryl Caprylate/Caprate is mild and substantially free of PEG, EO/PO, dioxane and Nitrogen. The multifunctional benefits include yield generation for suspension of particles at high temperature for product stability, viscosity modifier, enhanced preservation activity, scalp skin moisturization, wet and dry conditioning, and potential enhanced depo of soluble active. The glyceryl caprylate/caprate can be sourced from Stepan. Alternatively, the personal care composition can comprise from about 3 wt % to about 10 wt % Glyceryl Ester in combination with lauric acid. The lauric acid can be included in the personal care composition at from about 0.1 wt % to about 3 wt %, alternatively from about 0.1 wt % to about 2.5 wt %.

### Cationic Polymers

The personal care composition can include a cationic polymer. The cationic deposition polymer can be included at about 0.1 wt % to about 3 wt %, about 0.1 wt % to about 2.0 wt %, and at about 0.1 wt % to about 1.0 wt %. The cationic polymer can be a guar or a cellulose derivative.

The cationic deposition polymer can be included by weight of the personal care composition at about 0.1 wt % to about 3 wt %, about 0.1 wt % to about 2.0 wt %, and at about 0.1 wt % to about 1.0 wt %. The cationic deposition polymer can have a weight average molecular weight of from about 500 Thousand to about 2.2 Million, and from about 1.8 Million to about 2 Million; and a charge density of from about 0.4 to about 2.6, from about 0.4 to about 2, from about 0.7 to about 2, and from about 0.6 to about 1.6. Suitable polymers include KG 30 M and LR 30M available from Dow.

The charge densities can be measured at the pH of intended use of the personal care composition. (e.g., at about pH 3 to about pH 9; or about pH 4 to about pH 8).

In certain embodiments, suitable cationic polymers can contain cationic nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers so long as the polymers remain soluble in water, in the composition, and in a coacervate phase of the composition. Examples of suitable counterions include halide counterions (e.g., chloride, fluoride, bromide, iodide).

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers can include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers can include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). In certain embodiments, suitable cationic substituted monomers include cationic substituted dialkylaminoalkyl acrylamides, dialkylaminoalkyl methacrylamides, and combinations thereof. Generally, cationic monomers can conform to the formula III:

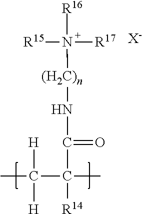

wherein R¹⁴ is hydrogen, methyl or ethyl; each of R¹⁵, R¹⁶ and R¹⁷ are independently hydrogen or a short chain alkyl having from 1 to 8 carbon atoms, or from 1 to 5 carbon atoms, or from 1 to 2 carbon atoms; n is an integer having a value of from 1 to 8, or 1 to 4; and X is a counterion. The nitrogen attached to R¹⁵, R¹⁶ and R¹⁷ can be a protonated amine (primary, secondary or tertiary), but can also be a quaternary ammonium wherein each of R¹⁵, R¹⁶ and R¹⁷ are alkyl groups. In certain embodiments, the cationic monomer can be polymethyacrylamidopropyl trimonium chloride, available under the trade name Polycare 133, from Rhone-Poulenc, Cranberry, N.J., U.S.A. In certain embodiment, copolymers of the cationic monomer are also suitable. In such embodiments, the charge density of the total copolymer can be from about 2.0 to about 4.5 meq/gm.

Other cationic polymers are also suitable including polysaccharide polymers, such as cationic cellulose derivatives and cationic starch derivatives. Suitable cationic polysaccharide polymers include those which conform to Formula IV:

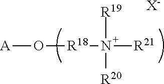

wherein A is an anhydroglucose residual group, such as a starch or cellulose anhydroglucose residual; R¹⁸ is an alkylene oxyalkylene, polyoxyalkylene, or hydroxyalkylene group, or combination thereof; R¹⁹, R²⁰, and R²¹ are independently alkyl, aryl, alkylaryl, arylalkyl, alkoxyalkyl, or alkoxyaryl groups, wherein each group contains up to 18 carbon atoms, and the total number of carbon atoms for each cationic moiety (i.e., the sum of carbon atoms in R¹⁹, R²⁰, and R²¹) is 20 or less; and X is an anionic counterion.

In certain embodiments, a cationic cellulose polymer can be selected from the salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable cationic cellulose polymers can include polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers can include cationic guar gum derivatives, such as guar hydroxypropyltrimonium chloride. Additional examples of suitable cationic guar gum derivatives can include the Jaguar series commercially available from Rhone-Poulenc Incorporated and the N-Hance series commercially available from Aqualon Division of Hercules, Inc. Additional details about cationic guar gum derivatives are disclosed in U.S. Pat. No. 6,930,078 which is incorporated by reference herein.

As can be appreciated, other cationic polymers known in the personal care industry are also suitable. For example, quaternary nitrogen-containing cellulose ethers are suitable. Examples of quaternary nitrogen-containing cellulose ethers are described in U.S. Pat. No. 3,962,418, which is incorporated herein by reference.

In certain embodiments, a synthetic cationic polymer or derivative thereof can be useful as a cationic polymer. Generally, such synthetic cationic polymers can be included at a concentration of from about 0.025% to about 5%, by weight of a personal care composition.

Suitable synthetic cationic polymers can include polymers which are water-soluble or dispersible, are cationic, and are non-crosslinked. In certain embodiments, suitable polymers can be conditioning copolymers comprising: (i) one or more cationic monomer units; and (ii) one or more nonionic monomer units or monomer units bearing a terminal negative charge; wherein said copolymer has a net positive charge. In certain embodiments, the synthetic cationic polymers can have a cationic charge density of from about 0.5 meq/g to about 10 meg/g, and can have an average molecular weight from about 1,000 to about 5,000,000.

Non-limiting examples of suitable synthetic cationic deposition polymers are described in United States Patent Application Publication No. US 2003/0223951 which is incorporated herein by reference.

Other suitable cationic polymers include copolymers of etherified cellulose, guar and starch, some examples of which are described in U.S. Pat. No. 3,958,581, which is incorporated herein by reference. Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

In embodiments including a cationic polymer, the cationic polymers can be soluble in the composition or can be soluble in a complex coacervate phase in the composition formed by interaction of the cationic polymer and a sulfate-free anionic, amphoteric or zwitterionic detersive surfactant. Complex coacervates of the cationic polymer can also be formed with other anionic charged materials in the personal care composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164 which is incorporated herein by reference.

### Anti-Dandruff Actives

The personal care composition also contains an anti-dandruff agent. Suitable anti-dandruff agents can include pyridinethione salts, azoles, selenium sulfide, particulate sulfur, and mixtures thereof. Such anti-dandruff particulate should be physically and chemically compatible with the essential components of the composition, and should not otherwise unduly impair product stability, aesthetics or performance In certain embodiments, a personal care composition can include a cationic polymer to enhance deposition of an anti-dandruff active. Particularly suitable antidandruff actives include zinc pyrithione, piroctone olamine, azoxystrobin, sulfur, salicylic acid and com.

a. Pyridinethione Salts

In certain embodiments, an anti-dandruff agent can be a pyridinethione particulate such as a 1-hydroxy-2-pyridinethione salt. The concentration of pyridinethione anti-dandruff particulates can range from about 0.1 wt % to about 4 wt %, about 0.1 wt % to about 3 wt %, about 0.1 wt % to about 2 wt % and from about 0.3 wt % to about 2 wt % by weight of the composition. Suitable pyridinethione salts include those formed from heavy metals such as zinc, tin, cadmium, magnesium, aluminum and zirconium, the zinc salt of 1-hydroxy-2-pyridinethione (known as “zinc pyridinethione” or “ZPT”), alternatively 1-hydroxy-2-pyridinethione salts in platelet particle form, wherein the particles have an average size of up to about 20 μ, alternatively up to about 5 μ, more alternatively up to about 2.5 μ. Salts formed from other cations, such as sodium, can also be suitable. Pyridinethione anti-dandruff agents are further described in U.S. Pat. Nos. 2,809,971; 3,236,733; 3,753,196; 3,761,418; 4,345,080; 4,323,683; 4,379,753; and 4,470,982, each of which are incorporated herein by reference. It is contemplated that when ZPT is used as the anti-dandruff particulate, that the growth or re-growth of hair may be stimulated or regulated, or both, or that hair loss may be reduced or inhibited, or that hair may appear thicker or fuller.

b. Other Anti-Microbial Actives

In addition to the anti-dandruff active selected from polyvalent metal salts of pyrithione, a personal care composition can further include one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Suitable anti-microbials can include itraconazole, ketoconazole, selenium sulphide and coal tar.

c. Azoxystrobin

The personal care composition may comprise from about 0.02% to about 10% of azoxystrobin; from about 0.05% to about 2% of azoxystrobin; from about 0.1% to about 1% of azoxystrobin.

d. Piroctone Olamine

Piroctone olamine can be used as an anti-microbial/anti-dandruff agent. Effective concentrations of the piroctone olamine are typically from about 0.1 wt % to about 2 wt %.

e. Salicylic Acid

Salicylic acid can be used as an anti-microbial/anti-dandruff agent. Effective concentrations of the salicylic acid are typically from about 0.1 wt % to about 10 wt %.

f. Azoles

In certain embodiments, a suitable anti-microbial agent can be an azole. Examples of azole anti-microbials can include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in a personal care composition, the azole anti-microbial active can be included in an amount from about 0.01% to about 5%, from about 0.1% to about 3%, and more from about 0.3% to about 2%, by weight of the composition.

g. Selenium Sulfide

Selenium sulfide is a particulate anti-dandruff agent suitable for use as an anti-microbial compositions when included at concentrations of about 0.1% to about 4%, by weight of the composition, from about 0.3% to about 2.5% by weight, and from about 0.5% to about 1.5% by weight. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, alternatively less than 10 μm as measured by forward laser light scattering device (e.g. Malvern 3600 instrument). Selenium sulfide compounds are described, for example, in U.S. Pat. Nos. 2,694,668; 3,152,046; 4,089,945; and 4,885,107, each of which are incorporated herein by reference.

h. Sulfur

Sulfur can also be used as a particulate anti-microbial/anti-dandruff agent in certain embodiments. Effective concentrations of the particulate sulfur are typically from about 0.1 wt % to about 5 wt %, from about 1 wt % to about 4 wt %, and from about 2 wt % to about 4 wt %.

k. Other

Additional anti-microbial actives can include extracts of melaleuca (tea tree) and charcoal. As can be appreciated, personal care compositions can also include combinations of anti-microbial actives. Suitable combinations can include octopirox and zinc pyrithione combinations, pine tar and sulfur combinations, salicylic acid and zinc pyrithione combinations, octopirox and climbasole combinations, and salicylic acid and octopirox combinations, and mixtures thereof.

### Humectant

A personal care composition can also include a humectant to lower the rate of water evaporation. Suitable humectants can include polyhydric alcohols, water soluble alkoxylated nonionic polymers, and mixtures thereof. The humectants, when included, can be used at levels by weight of the composition of from about 0.1% to about 20%, and from about 0.5% to about 5%.

Suitable polyhydric alcohols can include glycerin, sorbitol, propylene glycol, butylene glycol, hexylene glycol, ethoxylated glucose, 1, 2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Suitable water soluble alkoxylated nonionic polymers can include polyethylene glycols and polypropylene glycols having a molecular weight of up to about 1000 such as those with CTFA names PEG-200, PEG-400, PEG-600, PEG-1000, and mixtures thereof.

### Other Optional Components

As can be appreciated, a personal care composition can include still further optional components. For example, amino acids can be included. Suitable amino acids can include water soluble vitamins such as vitamins B1 , B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

In certain embodiments, a personal care composition can optionally include pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The compositions can also include antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

In certain embodiments, one or more stabilizers and preservatives can be included. For example, one or more of trihydroxystearin, ethylene glycol distearate, citric acid, sodium citrate dihydrate, a preservative such as kathon, sodium chloride, sodium benzoate, sodium salicylate and ethylenediaminetetraacetic acid (“EDTA”) can be included to improve the lifespan of a personal care compositon. The stabilizer and/or preservative can be used at a level of from about 0.10 wt % to about 2 wt %. Particularly suitable is sodium benzoate at a level of from about 0.10 wt % to about 0.45 wt %. The personal care composition may also include citric acid at a level of from about 0.5 wt % to about 2 wt %. The sodium benzoate and the citric acid can be added to the personal care composition alone or in combination.

Chelants can also be included to scavenge metal and reduce hair damage caused by exposure to UV radiation. Examples of suitable chelants can include histidine and N,N′ ethylenediamine disuccinic acid (“EDDS”).

Perfumes can also be included, and the perfume can be an essential oil.

### Method of Use

The personal care compositions described herein can be used in a conventional manner for cleansing and conditioning of hair or skin. Generally, a method of treating hair or skin can include applying the personal care composition to the hair or skin. For example, an effective amount of the personal care composition can be applied to the hair or skin, which has been wetted with water, and then the composition can be rinsed off. Effective amounts can generally range from about 1 g to about 50 g in certain embodiments, and from about 1 g to about 20 g in certain embodiments. Application to the hair typically includes working the composition through the hair such that most or all of the hair is contacted with the composition.

In certain embodiments, a method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

In certain embodiments, a personal care composition as described herein can be used to treat damaged hair. Damaged hair can include hair permed hair, oxidatively colored hair, and mechanically damaged hair.

The personal care compositions can be used as liquids, solids, semi-solids, flakes, gels, in a pressurized container with a propellant added, or used in a pump spray form. The viscosity of the product may be selected to accommodate the form desired.

### TEST METHODS

### Preparation of the Example Cleaning Compositions

The example cleaning compositions are prepared by combining the surfactant(s), the surfactant-soluble active, and the remainder of the water with ample agitation to ensure a homogenous mixture. The mixture can be heated to 40-70° C. to speed the solubilization of the surfactant-soluble agents, then cooled. Product pH is then adjusted as necessary to create a resultant composition of about 5 to about 6.

### Methods

pH

Utilized a Thermo Scientific Orion Star A321 pH meter. Calibration is to be done prior to pH analysis. After calibration pH measurements are made on fished product until desired pH value is obtained.

### Stability Assessment

Stability can be assessed by placing shampoo samples in a 40° C. temperature-controlled room. Changes in visual physical properties over time are assessed via observed phase separation or precipitation. A Yes or No result is given for samples placed within the stability conditions for up to 1 month. A Yes result means the product is phase stable with no visible signs of any of the characteristics of phase instability including creaming, precipitation, tunneling, separation, or sedimentation. A No result means that there is a visible sign of creaming, precipitation, tunneling, phase separation, or sedimentation that occurred at that 1st month of stability assessment.

### Rheology Measurement

Low shear yield stress is determined via Trios Rheometer at 26.7 C with 60 mm 2.005 degree cone plate, Peltier Aluminum plate. Sample gap set at 52 um. Sample is run via a logarithmic sweep from 10 to 1.0e−4 1/S with. Analysis on the Shear stress (Pa) curve from 10e−4 to 10e−2 is performed via the Herschel Bulkley analysis to determine the low shear yield stress in Pascals (Pa)

### Viscosity Measurement

Shampoo viscosities can be measured on a 2.5 mL sample using a cone and plate Brookfield RS rheometer with cone C75-1 at constant shear rate of 2 s−1, at 27° C. at 3 mins.

### Conditioning (Combing Friction and Combing Detangling)

Hair that had not previously been exposed to chemical treatments such as coloring or perming was used to measure the conditioning delivered by the formulated shampoos with added oils. Tresses (4 g, 8 inches) were washed for 3 cycles and the combing forces measured using an instron. The force required to pull the hair length through the comb was measured as Combing Friction and the force required to pull the hair tips through the comb was measured as Combing Detangling.

A lower force equals higher conditioning and less breakage. All the shampoos with the triglyceride oils show a significantly lower combing force than the no oil shampoo and are close to dimethicone conditioning performance. The compositions of these four oils cover the range of triglycerides typically found in natural oils from coconut oil which contains mainly short chain saturated triglycerides (fatty acid chain length C10, C12) to safflower seed oil and argan oil which contain mainly triglycerides with C18 fatty acids with either one or two double bonds and meadowfoam seed oil which contains longer chain unsaturated fatty acids (C20-C22).

Jojoba seed oil does not contain triglycerides, 80% of the oil is a straight-chain ester with 40-42 carbon atoms. Although shows a lower detangling force than the control shampoo with no oil it is not as low as the triglyceride oils and no combing friction benefit is measured.

### Cross Polar Microscopy/Light Microscopy

Samples are examined via DIC and cross-polar transmitted light microscopy using various objectives. Included images used a 40× objective. Light microscopy images at 40× taken with microscopy settings via DIC transmitted light with condenser setting II, Wallaston Filter in, Turet 2 settings.

pH

Utilized a Thermo Scientific Orion Star A321 pH meter. Calibration is to be done prior to pH analysis. After calibration pH measurements are made on fished product until desired pH value is obtained.

### NON-LIMITING EXAMPLES

The personal care compositions illustrated in the following Examples illustrate specific embodiments of the personal care compositions described herein, but are not intended to be limiting thereof. Other modifications can be undertaken by the skilled artisan without departing from the spirit and scope of this invention. These exemplified embodiments of the personal care composition provide suitable cleaning benefits to hair without the use of a harsh sulfate-based surfactant.

The personal care compositions illustrated in the following Examples are prepared by conventional formulation and mixing methods, an example of which is set forth below. All exemplified amounts are listed as weight percent's and exclude minor materials such as diluents, preservatives, color solutions, imagery ingredients, botanicals, and so forth, unless otherwise specified. All percentages are based on weight unless otherwise specified

Combination of ingredients including Sulfate free Sodium Cocoyl Alaninate surfactant and glyceryl caprylate caprate allows for a stable suspension of a high level of conditioning oils in formulation. These oils provided reduced hair surface friction parity to or significantly better than Silicone containing formula or no oil.

| | | | | | | | | | | | Com- | Com- | Com- |
|---|---|---|---|---|---|---|---|---|---|---|---|---|---|
| | | | | | | | | | | | parable | parable | parable |
| | 1 | 2 | 3 | 4 | 5 | 6 | 7 | 8 | 9 | 10 | 1. | 2. | 3. |
| Sodium Cocoyl | 9.2 | 14 | 14 | 14 | 14 | 14 | 14 | 14 | 14 | 14 | | 16 | 16 |
| Alaninate¹ | | | | | | | | | | | | | |
| Glyceryl Caprylate/ | 5 | 5.5 | 5.5 | 5.5 | 5.5 | 5.5 | 5.5 | 5.5 | 5.5 | 5.5 | 5.5 | 5 | 5 |
| Caprate2 | | | | | | | | | | | | | |
| Sodium Laureth 3 | | | | | | | | | | | 14 | | |
| Sulfate3 | | | | | | | | | | | | | |
| Lauramidopropylbetaine, | | | | | | | | | | | | | 6 |
| 4 | | | | | | | | | | | | | |
| Polyquaternium-10 | 0.6 | | | | | | | | | | | | |
| (KG30M)5 | | | | | | | | | | | | | |
| Polyquaternium-10 | | 0.6 | 0.6 | 0.6 | 0.6 | 0.6 | 0.6 | 0.6 | 0.6 | 0.6 | 0.6 | 0.6 | 0.6 |
| (JR30M)6 | | | | | | | | | | | | | |
| Polyquaternium-10 | | | | | | | | | | | | | |
| (LR30M)7 | | | | | | | | | | | | | |
| Safflower Oil, 8 | 2 | | | 2 | | | | | | | 2 | | |
| Meadowfoam Oil, 9 | | | | | 2 | | | | | | | | |
| Coconut Oil, 10 | | | | | | 2 | | | | | | | |
| Jojoba Oil, 11 | | | | | | | 2 | | | | | | |
| Argan Oil, 12 | | | | | | | | 2 | | | | | |
| Shea Butter 13 | | | | | | | | | 2 | | | | |
| Coco butter14 | | | | | | | | | | 2 | | | |
| Silicone 1872 | | | 2 | | | | | | | | | | |
| microemulsion, 15 | | | | | | | | | | | | | |
| Perfume, 16 | 1 | 1 | 1 | 1 | 1 | 1 | 1 | 1 | 1 | 1 | 1 | 1 | 1 |
| Sodium Benzoate, 17 | 0.25 | 0.25 | 0.25 | 0.25 | 0.25 | 0.25 | 0.25 | 0.25 | 0.25 | 0.25 | 0.25 | 0.25 | 0.25 |
| Sodium Chloride, 18 | 2 | 2.5 | 2.5 | 2.5 | 2.5 | 2.5 | 2.5 | 2.5 | 2.5 | 2.5 | 2.5 | | |
| Citric Acid, 19 | 1.6 | 1.6 | 1.6 | 1.6 | 1.6 | 1.6 | 1.6 | 1.6 | 1.5 | 1.7 | 1.6 | 1.6 | 1.6 |
| pH | 5.5 | 5.1 | 5.1 | 5.1 | 5.1 | 5.1 | 5.1 | 5.1 | 5.0 | 5.04 | 3.19 | 5.1 | 5.1 |
| Water, 20 | Q.S. | Q.S. | Q.S. | Q.S. | Q.S. | Q.S. | Q.S. | Q.S. | Q.S | Q.S | QS | QS | Q.S. |
| 1 m Visual Stability | Yes | Yes | Yes | Yes | Yes | Yes | Yes | Yes | Yes | Yes | No | No | No |
| (40 C.) | | | | | | | | | | | | | |
| Hershel Bulkey Low | 20 | 5.3 | 4.1 | 5.9 | 5.3 | 6.8 | 5.3 | 5.6 | 6.34 | 7.39 | 0.56 | NA | NA |
| shear Yield stress (Pa) | | | | | | | | | | | | | |

Ingredient Key

| | |
|---|---|
| 1. | Sodium Cocoyl Alaninate, Eversoft ACS (contains ~1% NaCl). |
| | Supplier: Sino Lion |
| 2. | Glyceryl Caprylate/Caprate, Stepan Mild GCC, containing |
| | mono, di, tri glycerides. Supplier: Stepan Company |
| 3. | Sodium Laureth Sulfate from P&G Chemicals |
| 4. | Lauramidopropylbetaine from Solvay |
| 5. | Polyquatemium10 KG30M from Dow |
| 6. | Polyquatemium 10 JR30M from Dow |
| 7. | Polyquatemium10 LR30M from Dow |
| 8. | safflower oil from Arista |
| 9. | Meadowfoam oil from Croda |
| 10. | Coconut oil from Southern Cross Botanicals |
| 11. | Jojoba oil from Southern Cross Botanicals |
| 12. | Argan oil from Carrubba |
| 13. | Shea Butter, Harris & Ford LLC |
| 14. | Cocoa Butter from The Hallstar Co. |
| 15. | Dimethyl silicone, Xiameter Silicone 1872 |
| | microemulsion from Dow |
| 16. | Sodium benzoate from Kalama Chemical |
| 17. | Perfume from Givuadan |
| 18. | Sodium Chloride from Morton |
| 19. | Citric acid from Archer Daniels Midland |
| 20. | DI Water and Minors (QS to 100%) from |
| | Misty Mountain Spring water |

| | 11 | 12 | 13 | 14 | 15 | 16 | 17 | 18 | 19 | 20 | 21 |
|---|---|---|---|---|---|---|---|---|---|---|---|
| Sodium Cocoyl | 12 | 12 | 9.0 | 9.0 | 14 | 14 | 14 | 12 | | | |
| Alaninate, 1 | | | | | | | | | | | |
| Disodium Cocoyl | | | | | | | | | 14 | | |
| Glutamate, 2 | | | | | | | | | | | |
| Sodium Lauryl | | | | | | | | | | 14 | |
| Sarcosinate, 3 | | | | | | | | | | | |
| Sodium Cocoyl | | | | | | | | | | | 14 |
| Glycinate. 4 | | | | | | | | | | | |
| Glyceryl Caprylate/ | 5 | 5.5 | 5.5 | 5.5 | 5.5 | 5.5 | 5.5 | 5.5 | 5.5 | 5.5 | 5.5 |
| Caprate, 5 | | | | | | | | | | | |
| Lauramidopropylbetaine, | 4 | 4 | 2 | 5 | | 4 | | 4 | 2 | 2 | 2 |
| 6 | | | | | | | | | | | |
| Polyquaternium-10 | | | | | | | | | 0.6 | 0.6 | 0.6 |
| (KG30M), 7 | | | | | | | | | | | |
| Polyquaternium-10 | 0.6 | 0.6 | 0.6 | 0.6 | 0.6 | 0.6 | 0.6 | 0.6 | | | |
| (JR30M), 8 | | | | | | | | | | | |
| Polyquaternium-10 | | | | | | | | | | | |
| (LR30M), 9 | | | | | | | | | | | |
| Safflower Oil, 10 | | | 2 | | | | | | | | |
| Meadowfoam Oil, 11 | 2 | | | 2 | | | | | | | |
| Coconut Oil, 12 | | | 2 | | 2 | | | | | | |
| Jojoba Oil, 13 | | 2 | | | | 0.5 | | 2 | | | |
| Argan Oil, 14 | | | | | | | 2 | | 2 | 2 | 2 |
| Perfume, 15 | 1 | 1 | 1 | 1 | 1 | 1 | 1 | 1 | 1 | 1 | 1 |
| Sodium Benzoate, 16 | 0.25 | 0.25 | 0.25 | 0.25 | 0.25 | 0.25 | 0.25 | 0.25 | 0.25 | 0.25 | 0.25 |
| Citric Acid, 17 QS to | QS | QS | QS | QS | QS | QS | QS | QS | QS | QS | QS |
| target | | | | | | | | | | | |
| pH | 5.1 | 5.1 | 5.1 | 5.1 | 5.1 | 5.1 | 5.1 | 5.1 | 5.1 | 5.1 | 5.1 |
| Water, 18 | Q.S. | Q.S. | Q.S. | Q.S. | Q.S. | Q.S. | Q.S. | Q.S. | Q.S. | Q.S. | Q.S. |

Ingredient Key

| | |
|---|---|
| 1. | Sodium Cocoyl Alaninate, Eversoft ACS (contains ~1% NaCl). |
| | Supplier: Sino Lion |
| 2. | Disodium Cocoyl Glutamate Eversoft50SG from Sino Lion |
| 3. | Sodium Lauryl Sarcosinate from Croda Chemicals |
| 4. | Sodium Cocoyl Glycinate, from Innospec |
| 5. | Glyceryl Caprylate/Caprate, Stepan Mild GCC, containing |
| | mono, di, tri glycerides. Supplier: Stepan Company |
| 6. | Lauramidopropylbetaine from Solvay |
| 7. | Polyquaternium10 KG30M from Dow |
| 8. | Polyquaternium 10 JR30M from Dow |
| 9. | Polyquaternium10 LR30M from Dow |
| 10. | Safflower oil from Arista |
| 11. | Meadowfoam oil from Croda |
| 12. | Coconut oil from Southern Cross Botanicals |
| 13. | Jojoba oil from Southern Cross Botanicals |
| 14. | Argan oil from Carrubba |
| 15. | Perfume from Givuadan |
| 16. | Sodium benzoate from Kalama Chemical |
| 17. | Citric acid from Archer Daniels Midland |
| 18. | DI Water and Minors (QS to 100%) from |
| | Misty Mountain Spring water |

| | 22 | 23 | 24 | 25 | 26 | 27 | 28 | 29 | 30 | 31 | 32 | 33 | 34 | 35 |
|---|---|---|---|---|---|---|---|---|---|---|---|---|---|---|
| Sodium Cocoyl | 14 | 12 | 14 | 14 | 14 | 14 | 12 | 14 | 14 | 12 | 12 | 14 | 12 | 12 |
| Alaninate, 1 | | | | | | | | | | | | | | |
| Glyceryl Caprylate/ | 5 | 5.5 | 5 | 5.5 | 5 | 5 | 4 | 4 | 10 | 4 | 5 | 5.5 | 4 | 4 |
| Caprate, 2 | | | | | | | | | | | | | | |
| Lauramidopropylbetaine, | 2 | 4 | 2 | 2 | 2 | 3 | 4 | 2 | | 4 | | | 4 | 4 |
| 3 | | | | | | | | | | | | | | |
| Polyquaternium-10 | | | 0.3 | | | | | 1 | | | | | | |
| (KG30M), 4 | | | | | | | | | | | | | | |
| Polyquaternium-10 | | 0.6 | | | | | 0.6 | | | 0.6 | 0.6 | 0.6 | 0.6 | 0.6 |
| (JR30M), 5 | | | | | | | | | | | | | | |
| Polyquaternium-10 | 0.6 | | | 0.6 | 0.15 | | | | 0.6 | | | | | |
| (LR30M), 6 | | | | | | | | | | | | | | |
| Guar | | | | | | 0.6 | | | | | | | | |
| Hydroxypropyltrimonium | | | | | | | | | | | | | | |
| Chloride (Excel guar) | | | | | | | | | | | | | | |
| Safflower Oil, 7 | | | | | | | | | | | | | | |
| Meadowfoam Oil, 8 | | | | | | | | | | | | | | |
| Coconut Oil, 9 | | | | | | | | | 2 | | | | | |
| Jojoba Oil, 10 | | 2 | | | | | 2 | | | 2 | 2 | 2 | 2 | 2 |
| Argan Oil, 11 | 2 | | 2 | 2 | 2 | 2 | | 2 | | | | | | |
| Piroctone olamine, 12 | | | | | | | | | | 0.5 | | | | |
| Zinc Pyrithione, 13 | | | | | | | | | | | 1 | 1 | 1 | 2 |
| Perfume, 14 | 1 | 1 | 1 | | 1 | 1 | 1 | 1 | 1 | 1 | 1 | 1 | 1 | 1 |
| Sodium Benzoate, 15 | 0.25 | 0.25 | 0.25 | 0.25 | 0.25 | 0.25 | 0.25 | 0.25 | 0.25 | 0.25 | 0.25 | 0.25 | 0.25 | 0.25 |
| Citric Acid, 16 QS to | QS | QS | QS | QS | QS | QS | QS | QS | QS | QS | QS | QS | QS | QS |
| target | | | | | | | | | | | | | | |
| pH | 5.1 | 5.1 | 5.1 | 4.8 | 4.8 | 5.2 | 5.1 | 5.1 | 5.1 | 5.1 | 5.1 | 5.1 | 5.1 | 5.1 |
| Water, 17 | Q.S. | Q.S. | Q.S. | Q.S. | Q.S. | Q.S. | Q.S. | Q.S. | Q.S. | Q.S. | Q.S. | Q.S. | Q.S. | Q.S. |

Ingredient Key

| | |
|---|---|
| 1. | Sodium Cocoyl Alaninate, Eversoft ACS (contains 1~% NaCl). |
| | Supplier: Sino Lion |
| 2. | Glyceryl Caprylate/Caprate, Stepan Mild GCC, containing |
| | mono, di, tri glycerides. Supplier: Stepan Company |
| 3. | Lauramidopropylbetaine from Solvay |
| 4. | Polyquaternium10 KG30M from Dow |
| 5. | Polyquaternium 10 JR30M from Dow |
| 6. | Polyquaternium10 LR30M from Dow |
| 7. | Safflower oil from Arista |
| 8. | Meadowfoam oil from Croda |
| 9. | Coconut oil from Southern Cross Botanicals |
| 10. | Jojoba oil from Southern Cross Botanicals |
| 11. | Argan oil from Carrubba |
| 12. | Piroctone Olamine, from Clariant Specialty Chemicals |
| 13. | Zinc Pyrithione, from Lonza |
| 14. | Perfume from Givuadan |
| 15. | Sodium benzoate from Kalama Chemical |
| 16. | Citric acid from Archer Daniels Midland |
| 17. | DI Water and Minors (QS to 100%) from |
| | Misty Mountain Spring water |

| | | | Dry Conditioning | | | |
|---|---|---|---|---|---|---|
| | | | Measurements | | | |
| | | | Combing | | Combing | |
| | | | Friction (gf) | | Detangling | |
| Formula | | % | std error | | (gf) std error | |
| Code | Oil | Added | in parenthesis | | in parenthesis | |
| Test 1 | | | | | | |
| Example 2 | None | 0% | 15.96 | (0.95) | 1062 | (150) |
| Example 4 | Safflower Seed Oil | 2% | 12.50 | (0.58)* | 196 | (19)* |
| Example 8 | Argan Oil | 2% | 12.61 | (0.50)* | 217 | (28)* |
| Example 7 | Jojoba Bean Oil | 2% | 18.06 | (0.56) | 500 | (52)* |
| Example 5 | Meadowfoam | 2% | 13.37 | (0.33)* | 162 | (13)* |
| | Seed Oil | | | | | |
| Example 6 | Coconut Oil | 2% | 13.25 | (0.57)* | 189 | (26)* |
| Example 3 | Dimethicone | 2% | 10.45 | (0.25)* | 279 | (32)* |
| Test 2. | | | | | | |
| Example 2 | None | 0% | 48.96 | (0.25) | 278 | (6) |
| Example 9 | Shea Butter | 2% | 29.12 | (1.92) | 149 | (10) |
| Example 10 | Cocoa Butter | 2% | 30.79 | (2.93) | 149 | (14) |

*Significant vs control with no oil at >95% confidence (student t-test)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) about 9 wt % to about 16 wt % amino acid surfactant;
b) about 3 wt % to about 10 wt % glyceryl ester;
c) about 0.2 wt % to about 6 wt % triglyceride oil; and
d) a liquid carrier, wherein the personal care composition is substantially free of sulfated surfactants, has a pH of about 4.5 to about 6 and a yield stress of from about 0.5 Pa to about 10 Pa.

2. The personal care composition of claim 1, further comprising an antidandruff agent selected from the group consisting of pyridinethione salts, zinc pyrithione, azoles, selenium sulfide, particulate sulfur, coal tar, sulfur, whitfield's ointment, castellani's paint, aluminum chloride, gentian violet, piroctone olamine, ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, azoxystrobin and combinations thereof.

3. The personal care composition of claim 1, wherein the pH is from about 4.5 to about 5.5.

4. The personal care composition of claim 1, wherein the yield stress of from about 1 to about 10 Pa.

5. The personal care composition of claim 1, wherein the glyceryl ester is glyceryl caprate, glyceryl caprylate, or a mixture thereof.

6. The personal care composition of claim 5, wherein the glyceryl caprylate/caprate is substantially free of PEG, EO/PO, Nitrogen, dioxane and mixtures thereof.

7. The personal care composition of claim 1, further comprising from about 0.01 wt % to about 5 wt % of a zwitterionic surfactant.

8. The personal care composition of claim 1, further comprising from about 0.1 wt % to about 2.5 wt % lauric acid.

9. The personal care composition of claim 1, wherein the personal care composition is stable at both 5° C. and 40° C.

10. The personal care composition of claim 1, further comprising a cationic deposition polymer.

11. The personal care composition of claim 10, wherein the cationic deposition polymer is a guar derivative, a cellulose derivative or a mixture thereof.

12. The personal care composition of claim 10, wherein the cationic deposition polymer has a charge density of about 0.7 to about 2.0 meq/g, and a weight average molecular weight of from about 500,000 to about 2,000,000.

13. The personal care composition of claim 1, wherein the composition comprises less than about 3 wt % of inorganic salt.

14. The personal care composition of claim 1, comprising about 7% to about 15%, by weight, of the sodium cocoyl alaninate.

15. The personal care composition of claim 1, wherein the personal care composition is substantially free of silicones.

16. The personal care composition of claim 1, wherein the composition comprises about 0.10 to about 0.45 wt % of sodium benzoate.

17. The personal care composition of claim 1, wherein the composition comprises about 0.5 to about 2 wt % of citric acid.

18. A personal care composition consisting of:
water, sodium cocoyl alaninate, glyceryl caprylate/caprate, cationic polymer, sodium benzoate, citric acid, triglyceride oil, and at least one additional ingredient selected from the group consisting of sodium chloride, a perfume, a co-surfactant, and an anti-dandruff agent, wherein the total number of ingredients in the composition is 9 or less.
